SEQUENCE LISTING (6 sequences)

SEQ ID NO: 1 - DNA, 20 bp, Artificial sequence
  Synthetic primer
ccatgaatcactcccctgtg

SEQ ID NO: 2 - DNA, 19 bp, Artificial sequence
  Synthetic primer
ccggtcgtcctggcaattc

SEQ ID NO: 3 - DNA, 22 bp, Artificial sequence
  Synthetic primer
cagggtagtcgtcagtggttcg

SEQ ID NO: 4 - DNA, 20 bp, Artificial sequence
  Synthetic primer
ggcctctgcagcaccctatc

SEQ ID NO: 5 - DNA, 22 bp, Artificial sequence
  Synthetic primer
cctggaggctgcacgacactca

SEQ ID NO: 6 - DNA, 23 bp, Artificial sequence
  Synthetic primer
ccctcgtccacgtggcatctcga

(19) United States
(12) Patent Application Publication
(10) Pub. No.: US 2005/0287514 A1
(43) Pub. Date: Dec. 29, 2005

(54) Compositions and methods useful for HCV infection

(75) Inventor: Randal Byrn, Wayland, MA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Vertex Pharmaceuticals Incorporated, Cambridge, MA (US)

(21) Appl. No.: 11/001,686

(22) Filed: Dec. 1, 2004

Related U.S. Application Data
(60) Provisional application No. 60/526,411, filed on Dec. 1, 2003.

(51) Int. Cl. A01N 1/02; C12N 5/08

(52) U.S. Cl. 435/2; 435/370

(57) ABSTRACT

The present invention provides compositions comprising cells that can effectively produce HCV after HCV infection, compositions for culturing the cells, methods for making the composition and methods for infecting the cells in the composition with HCV. The present invention also provides methods for assaying HCV production and methods for evaluating compounds that affect the production of HCV.

The present application claims the benefit of priority of U.S. Provisional Application No. 60/526,411, which was filed Dec. 1, 2003. The entire text of the aforementioned application is incorporated herein by reference.

### BACKGROUND

1. Field of the Invention

The present invention is generally directed to compositions comprising cells that can effectively reproduce HCV and methods and compositions for making and using the same.

2. Background of the Related Art

Although Hepatitis C Virus (HCV) replicates robustly in infected human, a robust method of growing the virus in cultured cells has not been perfected. When infectious serum is used to infect cultured human liver cells in vivo, only small amounts of HCV are replicated which are only detectable by reverse transcriptase polymerase chain reaction (RT-PCR).

Attempts to infect cultured cells with HCV have been reported for peripheral blood mononuclear cells, human B and T cell lines, human hepatocyte lines, and primary human fetal and adult cells. However, the results reported to date have been disappointing. Often viral replication is so low that HCV produced from an infected population of cells can only be detected, if at all, with RT-PCR and then only low numbers of copies of HCV RNA can be observed. Further, the viral production is sporadic and not reproducible from well to well on the same or different days with the same virus and cells. Further still, it takes several days, even as much as a month after administering the virus to observe the peak of infection, e.g., lacovacci et al., Hepatology 26(5):1328-1337 (1997). These problems frustrate the identification and rapid screening of compounds that may be useful for treating patients suffering from HCV and/or for research relating to HCV infection.

WO 02/077206 describes a method for growing HCV in cultured cells. The method, however, has disadvantages. In the method disclosed in WO02/077206 the cells must be used soon after isolation. Therefore, only a single experiment may be done with cells from a single donor. From one donor, one set of cells may be obtained, and then one experiment may be done. Furthermore, the media used in the method is relatively complex.

Thus, there remains a continuing need for a method for infecting and replicating HCV in cell culture. There is also a need for quick and efficient methods for determining compounds which inhibit HCV production in culture. This application solves these problems by providing compositions comprising cells that can effectively reproduce HCV, methods for making the composition of cells, media for culturing cells, methods for infecting cells with HCV, methods for assaying HCV infection, and methods for evaluating the ability of a compound to affect the production of an HCV using the compositions and methods of this invention.

### SUMMARY OF THE INVENTION

The present invention provides methods for making compositions comprising high HCV producing culture cells. In one embodiment, the present invention provides compositions comprising cell mixtures comprising cells from the liver of a human aged three months or older after conception which have been cryopreserved and which can be efficiently and effectively infected with an HCV. The present invention provides compositions comprising cell mixtures comprising cells from the liver of a human aged three months or older after conception which can be efficiently and effectively infected with an HCV in a simple, hormonally defined media. The present invention also provides compositions comprising cells prepared by the methods of this invention. In another embodiment of this invention, the cells in the cell mixture can pass through a filter about 40 microns to about 70 microns in size. In another embodiment of this invention, the composition is used in conjunction with or further comprises a feeder cell. In yet another embodiment of this invention, the feeder cell is a STO(Reid-99) cell. STO(Reid-99) cells are merely exemplary feeder cells and other such feeder cells are readily available from the American Type Culture Collection.

The present invention provides compositions for culturing cells. In one embodiment of this invention, the compositions for culturing cells comprising a media, comprising: BSA nicotinamide, epidermal growth factor (EGF), insulin, transferrin and hydrocortisone.

The present invention provides methods for infecting a cell mixture by administering an HCV to compositions of this invention. According to one embodiment of this invention, the HCV is RNA898. In another embodiment of this invention, the HCV virus is initially incubated with the composition (innoculum) for about 4 to about 24 hours at about 37° C. in a volume of about 0.52 ml per cm² prior to washing the cells in the composition or replacing the innoculum with cell culture media.

The present invention provides a method for assaying HCV infection by incubating a composition of this invention with a feeder cell, contacting the cells in the composition with an HCV; and measuring the HCV associated with the cells and/or media in which the cells are cultured.

Further, the present invention provides a method for evaluating the ability of a compound to affect the production of HCV, i.e., affect the ability of the composition of cells to produce more HCV, comprising the steps of incubating a composition of this invention with a feeder cell, contacting the cells in the composition with an HCV virus and administering the compound before or after contact with HCV. In one embodiment, the method is used to screen for cells that inhibit HCV production. In a further embodiment, the method is used to screen a plurality of compounds simultaneously for their ability to inhibit HCV production.

In another embodiment, presence of HCV is determined by measuring the quantity of HCV RNA by reverse-transcriptase polymerase chain reaction (RT-PCR). In one embodiment, the HCV RNA in the sample is compared to an amount of RNA from a second virus that is used as an internal control. In a further embodiment, the second virus is the Bovine Viral Diarrhea Virus (“BVDV”).

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1 depicts cryopreserved human fetal liver cells after thawing and attachment to collagen-coated tissue culture wells. Cryopreserved fetal liver cells were thawed and cultured on collagen I coated tissue culture dishes in hormonally-defined medium 5 hours (FIG. 1A) or 24 hours (FIG. 1B) after attachment. Representative microscope fields are shown, 40× objective, Hoffman Differential optics.

FIG. 2 depicts a time course of increase of cell associated HCV RNA after infection of cryopreserved human fetal liver cells.

FIG. 3 depicts the inhibition of HCV infection of cryopreserved human fetal liver cells by the HCV NS3·4a protease inhibitor VX-950 (see WO 02/18369).

### DESCRIPTION OF THE PREFERRED EMBODIMENTS

There remains a need for cells that are suitable for in vitro growth of HCV. It would be most beneficial if liver cell populations can be produced that are able to be infected by HCV and yet can be produced in long-term in vitro cell culture and not have to be used immediately upon isolation. The present invention addresses these needs by providing a cell mixture that comprises liver cells and hematopoietic cells isolated from the liver of a human aged three months or older after conception. The mixed cell population preparation is such that 4×10⁴ cells of such a cell mixture grown in the presence of a feeder cell line in a growth media produces more than about 5000 copies of hepatitis C virus (HCV) RNA in the media seventy two hours after administration of, or infection with, a HCV virus, such as RNA898 to the cells.

A composition of this invention comprises a cell mixture comprising cells released from the liver of a human aged three months or older after conception that have been cryopreserved.

Also provided by this invention is a composition comprising a cell mixture comprising cells released from the liver of a human aged three months or older after conception which can be efficiently and effectively infected with an HCV in a simple, hormonally defined media.

Also provided by this invention is a composition comprising a cell mixture comprising cells released from the liver of a human aged three months or older after conception that have been cryopreserved and which can be efficiently and effectively infected with an HCV in a simple, hormonally defined media.

According to one embodiment, the human is aged between and including three months after conception up to 1 year after birth. In another embodiment of this invention, the human is aged three to six months after conception. In another embodiment, the human is aged between 18 to 22 weeks after conception. In one embodiment of this invention, the cells comprise fetal liver and hematopoietic cells. According to one embodiment of this invention, the liver and hematopoietic cells can express alpha fetoprotein, albumin and/or glycophorin. According to one preferred embodiment, if the human is an adult, the human liver is healthy.

The present invention includes a composition comprising cells which are significantly better host cells for the infection and replication of the HCV virus, RNA898 (hereinafter, “RNA898”). RNA898 was deposited on Mar. 27, 2001, in the American Type Culture Collection (“ATCC”), 10801 University Boulevard, Manassas, Va. 20110-2209) (ATCC Deposit No: PTA-3237) under the conditions of the Budapest Treaty. According to one embodiment, a composition of this invention is capable of producing more than about 5,000 copies; more than about 10,000 copies; or more than about 50,000 copies of hepatitis C viral RNA in the media seventy-two hours after administering the virus if there are 4×10⁴ cells in the composition. For example, a composition prepared according to the methods of this invention and assayed according to the methods herein would be capable of producing more than about 5,000, more than about 10,000 copies, or more than about 50,000 copies of hepatitis C viral RNA in the media seventy-two hours after administering the virus.

One of skill in the art would readily understand that if the number of cells in the composition were greater than 4×10⁴ cells, then the total number of copies of viral RNA being produced would be increased by an amount commensurate with the increased number of cells in the composition. Similarly, one of skill in the art would readily understand that if the number of cells in the composition were smaller than 4×10⁴ cells, then the total number of copies of viral RNA being produced would be decreased by an amount commensurate with the decreased number of cells in the composition. Accordingly, compositions comprising less or more than 4×10⁴ cells which would proportionally produce the same number of copies of HCV RNA are contemplated. The compositions according to this invention are capable of producing 5,000-55,000 copies of HCV RNA; 10,000-55,000 copies of HCV RNA and 25,000-55,000 copies of HCV RNA seventy-two hours after administration of the virus to the composition.

In certain exemplary embodiments, the present invention describes a cell mixture of compositions according to this invention can be prepared according to the steps that comprise obtaining a liver of a human aged three months or older after conception in a buffer comprising EGTA and incubating the dissected liver in compositions that will allow the cells to separate from the liver. The separated cells are treated so that objects about 40 micron or larger are removed from the separated cells. Additionally, red blood cells also are removed from the cell separated cells. The separated cells are then resuspended in a serum-free media comprising 0.1 mM to 0.6 mM calcium, bovine serum albumin, nicotinamide, epidermal growth factor (EGF), insulin, transferrin and hydrocortisone and cultured in serum free media. This final resuspension and culturing step may or may not be preceded by a step that involves cryopreservation of the separated cells by resuspending the same in a composition that comprises 10% DMSO and 10% fetal calf serum. In this manner a preferred population of cells of the present invention is prepared into a composition, wherein the compositions comprises liver cells and hematopoietic cells isolated from the liver of a human aged three months or older after conception, wherein a preparation that comprises 4×10⁴ cells of said cell mixture in the presence of a feeder cell line in a growth media produces more than about 5000 copies of hepatitis C virus (HCV) RNA in the media seventy two hours after administration of HCV virus RNA898 to said preparation. The preparation may or may not be cryopreserved. Methods and compositions for making and using such compositions are described in further detail below.

An initial step in the preparation of the cell population and compositions of the present invention involves obtaining an appropriate liver cell population. Preferably, the liver cell population includes cells that are primate, and most preferably, human cells. The human cells are preferably isolated from a human aged three months or older after conception. According to one embodiment, the human is aged between and including three months after conception up to 1 year after birth. In another embodiment of this invention, the human is aged three to six months after conception. In another embodiment, the human is aged between 18 to 22 weeks after conception. In one embodiment of this invention, the cells comprise fetal liver and hematopoietic cells. According to one embodiment of this invention, the liver and hematopoietic cells can express alpha fetoprotein, albumin and/or glycophorin. According to one preferred embodiment, if the human is an adult, the human liver is healthy.

Dissection of liver from various sources for the present invention typically is carried out in an ethylene glycol bis(β-aminoethyl ether)-N,N,N′,N′-tetraacetate (EGTA) buffer. In one embodiment, the EGTA buffer comprises 0.1 mM to 1.0 mM of EGTA. In another embodiment of this invention, the EGTA concentration is 0.5 mM. The organ material is dissected in a cold (4° C.) aliquot of this buffer. The buffer preferably contains an agent that facilitates the disaggregation of the organ tissue. Disaggregation may be facilitated by the presence of proteases. The protease solution may be warm or cold. Typically, the protease used is trypsin. One of the disadvantages of trypsinization is that that is may cause damage if the tissue is exposed to trypsin at elevated (e.g., room temperature or higher) for prolonged periods of time. Thus, cells are typically harvested within 30 minutes of incubation in warm trypsin. However, sufficient disaggregation of tissue typically requires 3 to 4 hours, and hence it is often desirable to perform the trypsinization at low (e.g., 4° C.) temperatures. This minimizes the damage to the cells while allowing the tissue to be appropriately soaked in the protease. In this manner, the tissue may be soaked in an EGTA buffer that contains 0.25% trypsin for between 10 minutes to up to 18 hours at 4° C. The tissue is then removed from the protease-containing buffer and placed in a 37° C. bath for 2-30 minutes. The cells are then separated from the tissue aggregate by adding e.g., 1 ml of preferably warm medium for every 100 mg original tissue present. Gentle pipetting of the mixture up and down facilitates the disaggregation of the tissue to allow the cells to become dispersed in the medium.

Instead of trypsin, or in addition to trypsin other proteases also may be used to facilitate disaggregation of the tissue. In preferred embodiments, the disaggregation is achieved using collagenase. Crude collagenase (e.g., 2000 units/ml) is used. Collagenase is preferred to trypsin because many tissues are less sensitive to collagenase than to trypsin and therefore damage to tissue cell integrity is less likely with collagenase. In a preferred aspect, the method of disaggregation involves a two step liver perfusion using a first incubation step in EGTA followed by a second incubation in calcium-containing buffer that contains collagenase (Seglen, P.O. Methods Cell Biol 13:29, 1976). Tissue is chopped in an EGTA buffer. The EGTA buffer is then removed and replaced with an alternate buffer or media that contains collagenase and calcium but does not contain the chelating agent (i.e., EGTA or equivalent thereof), that contains e.g., 200 units collagenase/ml. The tissue is then incubated in this solution at e.g., 37° C. for e.g., for 10 minutes to up to 48 hours without agitation. Effective disaggregation is discernable if the tissue smears the bottom of the vessel in which it is being incubated. After the tissue has disaggregated the mixture is centrifuged at 50-100 g for 3 minutes. The supernatant is discarded and the cells are resuspended in an appropriate medium and cultured to expand the cells. In specific and preferred embodiments, the disaggregation is carried out using a collagenase buffer that comprises 0.1 to 5.0 mg/ml of collagenase. In another embodiment of this invention, the concentration of the collagenase is 2 mg/ml. (supplier: GIBCO TNVITROGEN, e.g., liver perfusion medium is obtained from GIBCO Cat. No. 17701). In preferred disaggregation methods, the tissue is incubated in EGTA for 10 minutes. The tissue is allowed to settle and the supernatant containing the EGTA is removed. The sedimented tissue is then incubated in buffer containing collagenase (e.g., GIBCO liver perfusion medium Cat. No. 17701) for 30 minutes.

Additional proteases that may be used include bacterial proteases such as for example, pronase (Schaffer et al., Am. J. Physiol., 273(3,1)G686-G695, 1997; Glavin et al., J. Pharmacol. Exp. Therapeut. 276:1174-1179, 1996) and dispase (Compton et al., J. Cell. Physiol., 177:274-281, 1998; Inamatsu et al., J. Invest. Dermatol., 111:767-775, 1998). In addition, other enzymes such as hyaluronidase and neuraminidase may also be used.

Once the disaggregation step is completed the cell suspension may be further processed to remove objects and materials. In particular and preferred embodiments, the cell population of the invention can pass through a filter about 40 microns in size. Thus, one of the steps of the preparation of the cell population of the invention involves removing objects from the disaggregated tissue that are larger than 40 microns. This size exclusion step according to this invention is meant to remove objects such as tissue, debris and aggregates of cells which cannot pass through a filter of about 40 microns in size. Thus, for example, the use of filters approximately 40 microns in size up to 100 microns in size (e.g., 50, 60, 70, 80, or 90 microns) and other methods for removing debris greater than 40 microns in size are contemplated. In one embodiment, the filtration step removes objects that cannot pass through a filter that is greater than about 40 microns in size. Multiple filtration steps may be employed e.g., an initial filtration step using a larger pore filter may be followed by a subsequent filtration step using a smaller pore size. Examples of filters according to this invention include nylon filters (e.g., “Cell strainer,” from Falcon (catalogue nos. 2034, 2350 or 2360)).

In addition to removing objects larger than 40 microns from the cell population, the separation methods of the invention also include the step of removing red blood cells from the cell mixture. It should be understood that the red blood cells can be removed at any stage during the preparation process after the cells are separated from the liver. Methods for removing red blood cells are known in the art. According to one embodiment of the invention, the red blood cells are removed by successive low speed spins in a centrifuge. For example, the separated cells that were passed through the filters can be spun at 50×g (450 rpm) for 4 minutes, the cell pellet can be resuspended and the same process repeated several times.

In this manner a cell population is obtained that is a suspension of liver cells from a human aged three months or older after conception, wherein the suspension of liver cells is comprised of cells that are smaller than 40 microns. Preferably, the suspension is one which is substantially free of red blood cells.

The cell population of the present invention is one which is enriched in cells that express alpha fetoprotein, albumin and/or glycophorin. Preferably, the cells are ones which do not express CD34 (and as such are referred to as CD34⁻). Those of skill in the art are aware of techniques for assessing whether the cells of the population contain one or more of these markers. Such techniques may involve immunostaining. For example, the anti-alpha fetoprotein antibodies from DAKO Corporation, Carpinteria, Calif., the anti-glycophorin antibodies (32591) from PharMingen, San Diego, Calif., the anti-human CD34 antibodies (34371A) from PharMingen, San Diego, Calif. and the anti-albumin antibodies (YM5024) from Accurate Chemical Corp., Westbury, N.Y. can be used.

In addition to determining the presence of these markers on the surface of the isolated cell population using the above-discussed antibodies, those of skill in the art will understand that it is possible to use such antibodies in isolation techniques to isolate the desired cell population. Thus, in exemplary embodiments, the primary cells isolated by dissecting a liver of a human aged three months or older after conception as described above are expanded in culture, either alone or in the presence of a feeder cell line and the expanded culture is enriched for the presence of cells that will be useful for the present invention. Such enrichment techniques may advantageously employ techniques such as immunologically-based procedures. These are comprised of, but not limited to, immune adhesion, fluorescence-activated flow cytometry, immunological-based column chromatography, antibody-conjugated sepharose beads (or other inert beads), or other immunology based applications (e.g. immuno-magnetic separation). These procedures do not however, define the population of liver cells, but rather lead to its isolation.

Other physical separation procedures may be applied prior to or after the antigenic purification. These are comprised of, but not limited to, equilibrium density centrifugation, velocity sedimentation, or counter-flow centrifugal elutriation. As well, other antigenic markers may be used in a positive or negative aspect, further define these cells. These are comprised of, but not limited to, antigens of the animal major histocompatibility locus (particularly HLA-DRA), hematopoietic antigens (e.g., CD33, CD8, CD10, CD14, CD9, CD20), or other liver proteins.

Liver cells of the invention can be enriched by equilibrium-density centrifugation of cells. Equilibrium-density centrifugation of the cells provides low density cells somewhat enriched in appropriate cells. Ficoll or Percoll densiy gradients may be used. In one embodiment, equilibrium-density centrifugation can be performed before the immunoaffinity step. In this embodiment, the antibody purification step is carried out on liver cells with a desired density. In a further embodiment, equilibrium-density centrifugation can be performed after the antibody purification of cells. Alternatively, the equilibrium-density centrifurgation purification step can be performed twice—once before the antibody purification, and once after the antibody purification step.

In another aspect, the population of liver cells can be enriched by using adherence to plastic. The preferred population of the present invention are plastic-adherent liver cells. The population of liver cells can be enriched by removing non-adherent cells present in the isolated population of cells. Removal of these non-adherent cells can be accomplished by exposing the liver cell population to an adherent surface, typically tissue culture plastic or glass. The adherent liver cells adhere to tissue culture plastic or glass while the non-adherent cells remain in suspension. The cells in suspension can easily be removed by removing the supernatant and washing the adherent cells. The non-adherent cells can be removed before or after the immunopurification step. Preferably, stromal cells are removed prior to the immunopurification step. The use of solid surfaces such as tissue culture plastic or glass is well known in the art. Tissue culture plastic and glass can be treated (e.g. silicone, nitrocellulose, nickel, lysine, etc.) to promote or inhibit cell adhesion. Treated and untreated surfaces are available commercially.

In another aspect, an enriched population of cells further is fractionated according to size. In a preferred embodiment, size fractionation can be accomplished by fluorescence activated cell sorting (FACS) using e.g., a FACScan flow cytometer (Becton Dickinson). Cells of the present invention have average diameters that are less than 40 microns, and more preferably are from about 10 microns to about 35 microns in diameter.

FACS permits the separation of sub-populations of cells on the basis of their light scatter properties as they pass through a laser beam. The forward light scatter (FALS) is related to cell size, and the right angle light scatter, also known as side scatter characteristic (SSC) to cell density, cellular content and nucleo-cytoplasmic ratio, i.e. cell complexity. Since cells can be labeled with fluorescent-conjugated antibodies, they can further be characterized by antibody (fluorescence) intensity. In exemplary embodiments, the FACS machine can be set to separate CD34⁻ (low fluorescence) and CD34⁺ (high fluorescence) cells.

In order to label liver cells with an antibody, for example a CD34⁺ monoclonal antibody, cells may be prepared by dissecting them from liver and treating them with protease as described in Example 1. The cell preparation is divided into aliquots comprising about for example 1×10⁵ to 1×10⁶ cells per ml in a centrifuge tube. This suspension is then centrifuged in a bench top or other centrifuge. The cell pellet thus generated is then resuspended in a suitable buffer that contains the monoclonal antibody of choice and allowed to incubate for an appropriate period of time. The labeled cells are washed and an aliquot of the labeled cells is incubated with fluorescein-labeled anti-mouse immunoglobulins. This suspension is washed and resuspended in a suitable buffer for cell sorting.

In sorting cells by FACS, a window (i.e., an electronically defined region) for low SSC and intermediate size (FALS) is set. The cells in this window are further divisible by antibody-fluorescence activity. The FACS settings are calibrated to collect cells positive and negative for the particular antigen.

In particular embodiments, of the present invention the liver cells may be characterized by FACS. The above technique for identifying and isolating CD34⁻ cells is repeated for the other antigenic determinants using the specific antibodies, such as those exemplified above. In this manner the skilled artisan is able to produce a highly enriched population of cells of the present invention.

As an alternative to FACS, cells may be isolated using immune-isolation. The availability of antibodies against the specific antigens of interest allows for the specific separation of liver cells of interest. In such embodiments, CD34⁺ cells are separated from the CD34⁻ cells and the CD34⁻ cells are then further separated according to the presence of other antigenic determinants on the cells (e.g., using anti-alpha fetoprotein antibodies; anti-glycophorin antibodies; anti-albumin antibodies and the like). Purification techniques are well known to those of skill in the art. These techniques tend to involve the dissection and fractionation of the cellular milieu from the liver to separate the liver cell fraction containing the desired cell from other components of the mixture using standard immunological procedures such as, but not limited to, immunomagnetics, immunoadhesion, and the like. Having separated desirable liver cell fraction as described above, the cells may be purified further using various separative to achieve further purification. Analytical methods particularly suited to the preparation of a pure cell surface antigens use chromatography.

Affinity chromatography is a chromatographic procedure that relies on the specific affinity between a substance to be isolated and a molecule that it can specifically bind to. This is a receptor-ligand type of interaction. The column material is synthesized by covalently coupling one of the binding partners (in this case the antibody) to an insoluble matrix. The column material is then able to specifically adsorb the substance from the solution. Elution occurs by changing the conditions to those in which binding will not occur (alter pH, ionic strength, temperature, etc.).

One of the most common forms of affinity chromatography is immunoaffinity chromatography. The generation of antibodies that would be suitable for use in accord with the present invention is discussed below. The antibody or antigen is attached to an insoluble, inert matrix. The cell population to be separated is applied to the matrix such that there is a specific antibody-antigen interaction. The bound antigen/antibody is eluted from the adsorbent by exposure to mildly denaturing solvents, such as low pH buffers, high salt concentrations and the like.

In particular embodiments, the cells of the present invention, are isolated using immunomagnetic chromatography. For example, an anti-CD34 or other antibody is attached to magnetic beads. These antibody-labeled magnetic beads are used as the basis for the affinity purification. The antibody-labeled whole liver cell preparation of cells produces herein is applied to the e.g., anti-CD34 antibody containing magnetic affinity column. The non-adherent cells are collected and the adherent cells are discarded from the magnetic column by removal of the magnetic field. In another embodiment, the cells are first labeled with an antibody (e.g., anti-CD34 antibody) and then labeled with a secondary antibody carrying a magnetic bead or sphere.

Another type of affinity chromatography useful in the purification of carbohydrate containing compounds is lectin affinity chromatography, this type of chromatography will be useful in removing CD34⁺ cells. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

Immunoselection using magnetic beads employs beads that are precoated with the desired monoclonal antibody. Cells may be precoated with monoclonal antibody and attached to the magnetic bead through a secondary anti-mouse immunoglobulin attached to the bead. The cells bound to the beads are then removed with a magnet. This is a rapid method that allows for a high yield of desired cells.

In immune adhesion, plastic coated surfaces are employed to isolate cells from a mixture. A surface such as a petri dish is coated with an antibody that selects cells that possess a desired phenotype. Cells are placed onto the coated dish and incubated for a suitable period of time to allow for an interaction between the cells and the coating of the dish. After the incubation period non-adherent cells are removed by gentle washing with a suitable buffer. The antibody-adherent cells that remain attached to the dish may then be cultured and/or recovered using trypsinization or other cell harvesting techniques well known to those of skill in the art.

The desirable liver cell population of the present invention is one which is immunoreactive with antibodies directed to alpha fetoprotein, albumin, and glycophorin (i.e., the cells of the population are positive for, and contain each of these markers) but is not immunoreactive with anti-CD34 antibodies (i.e., does not contain CD34). Antibodies are thus used to enrich the population of liver cells. As the liver cells are further characterized, other antibodies which immunoreact with a liver cell may be generated by one of ordinary skill in the art. The use of these other antibodies immunoreactive with a liver cell antigenic determinant are contemplated as well.

In another embodiment, a second antibody immunoreactive with an antibody that is immunoreactive with a determinant on the liver cell can be used to enrich the population of liver cells. The use of a secondary antibody is generally known in the art. Typically, secondary antibodies are antibodies immunoreactive with the constant regions of the first antibody. Preferred secondary antibodies include anti-rabbit, anti-mouse, anti-rat, anti-goat, and anti-horse immunoglobulins and are available commercially. In a preferred embodiment, secondary antibodies are conjugated to a solid substrate including tissue culture dish, agarose, polyacrylamide, and magnetic particles. In this embodiment, the antibody specific for a determinant on the cell surface of the liver cell is first immunoreacted to a population of liver cells to be isolated. The liver cell population containing the cells with the attached antibody is then exposed to the secondary antibody that is conjugated to a solid substrate. Enrichment of cells is achieved because only cells that are labeled with an antibody immunoreact with the secondary antibody. Commercially kits are available that provide secondary antibodies conjugated to magnetic particles. In this system, the appropriately labeled liver cells that present an antibody are purified by exposure to a magnetic field.

In specific embodiments, the cells of the invention are cryopreserved. Methods of cryopreservation of hepatocytes are known to those of skill in the art, see e.g., Mitry et al., Cell & Developmental Biology 13, 463-467 (2002); see also U.S. Pat. No. 5,723,282; U.S. Pat. No. 6,521,402 and particularly, U.S. Pat. No. 6,136,525 (incorporated herein by reference in its entirety) which describes certain cryoprotective media for cryopreservation of liver cells. In the present application, the cells to be cryopreserved are resuspended in a medium that contains a final concentration of 10% DMSO and 10% fetal calf serum. The cells are then dispensed into freezer safe containers, and the containers are frozen for a particular period of time under particular conditions, nitrogen, either liquid or vapor, storing said containers and thawing said cells when ready for use. Generally speaking, a cryoprotectant is a compound which is used to minimize the deleterious effects of cryopreservation such as the formation of intracellular ice during freezing. By way of illustration and not limitation mention may be made of DMSO, polyethylene glycol, amino acids, propanediol, etc.

A preferred hepatocyte culture medium is one which allows the cells to withstand the extreme temperature change encountered with liquid nitrogen storage with minimal cell degradation. In certain embodiments, the medium is a hormonally defined DMEM medium that contains BSA, nicotinamide, EGF, insulin, transferrin, and hydrocortisone (HDM2) and further may contain one or more supplements such as thymidine, arginine, insulin, dexamethasone, glutamine and mammalian serum. The following table provides exemplary concentrations of the medium that may be used and variations thereof.

| | | | Sources, alternatives and range of |
|---|---|---|---|
| | HDM2 | | concentrations |
| Medium | DMEM | | DMEM, High Glucose (JRH |
| | | | Biosciences, cat. No. 51444 is a |
| | | | preferred DMEM, however, RPMI |
| | | | 1640 or other similar buffer also |
| | | | may be used |
| BSA | 500 | ug/ml | Fatty acid free BSA is used (Sigma |
| supplemented | | | cat. no. A8806) supplemented with |
| with free | | | 7.6 uM free fatty acids according to |
| fatty acids | | | Cheesebauf and Padieu, In Vitro |
| | | | 20: 780, 1984. The range of free |
| | | | fatty acids may be varied from about |
| | | | 200 ug BSA/ml to about 2000 ug/ml |
| Nicotinamide | 2.5 | mM | Sigma, Cat. No. N0636. The range |
| | | | of nicotinamide may be varied from |
| | | | about 0.5 mM to 5 mM |
| EGF | 100 | ng/ml | In a preferred preparation, |
| | | | recombinant human EGF from |
| | | | Peprotech, cat no 100-15 is used. |
| | | | The preferred range of EGF may be |
| | | | varied from about 10 ng/ml to about |
| | | | 500 ng/ml |
| Insulin | 10 | ng/ml | (Sigma, cat. no. I5500). The |
| | | | preferred range of insulin may be |
| | | | varied from about 1 ng/ml to |
| | | | 10 ug/ml |
| Transferrin | 5 | ug/ml | The preferred transferrin |
| | | | composition used is human |
| | | | holotransferrin (available from |
| | | | Sigma cat. no. T0665). The |
| | | | preferred range of transferring may |
| | | | be varied from about 2 ug/ml to 20 |
| | | | ug/ml |
| Hydrocortisone | 10⁻⁶ | M | Hydrocortison is available from |
| | | | Sigma, hydrocortisone 21 |
| | | | hemisuccinate, cat. no. H4881. The |
| | | | preferred range of hydrocortison is |
| | | | from about 10⁻⁵ M to 10⁻⁷ M |

Mammalian serum may comprise fetal bovine serum, fetal calf serum and porcine serum in concentrations ranging from 10% to 90%. In particularly preferred embodiments, the medium is additionally supplemented with DMSO ranging from about 10 to about 15%, albumin ranging from about 3.5 to about 15% and glycerol ranging from about 10 to about 20%. In specific embodiments, the medium for cryopreservation comprises Fetal Bovine Serum, hereinafter FBS, plus Dimethyl Sulfoxide, hereinafter DMSO. More specifically the cryoprotectant medium comprises from about 5 to about 20% FBS and from about 5 to about 15% DMSO. Most preferred is a cryoprotectant medium comprising 10% FBS and 10% DMSO.

The isolated liver cell population defined above is prepared for cryopreservation by dispensing into freezer resistant containers at particular densities. Such containers include but are not limited to vials, bags, canes, etc. Preferred are plastic bags and most preferred are Cryocyte trademark of Baxter plastic bags having a capacity ranging from about 250 ml to about 500 ml.

Cells are dispensed into the containers and the containers are sealed using e.g., mechanical aluminum seals, thermal impulse heat sealers, luer lock plugs, etc. Heat sealing being most preferred. The thus sealed containers are preferably kept at 0-4 degrees Celsius until all the containers to be cryopreserved are filled and sealed so that they may be cryopreserved simultaneously.

The cell densities of the cells being cryopreserved may vary. For example, the cells may be cryopreserved in a density of from about 5×10⁶ cells/ml to about to about 40×10⁶ cells/ml. Volumes ranging from about 10 to about 50 mls may be preserved in 250 ml freezing containers, while from about 50 to about 150 mls may be cryopreserved in 500 ml freezing containers. In addition, the cells may be seeded in order to produce a controlled crystallization or ice formation in solutions which have already been cooled below freezing. Methods of seeding are known to those of skill in the art and include inserting a cold metal rod into the freezing containers, introducing a blast of liquid nitrogen into the freezing containers, etc.

Uniform freezing of the cells is preferred and may be achieved using freezing plates in which the containers to be frozen are positioned between the freezing plates.

Once the liver cells have been dispensed in freezing containers they are ready to be placed in freezers. While any freezer capable of freezing from about 4° C. to about minus 90° C. is contemplated, a control rate freezer is preferred. When using a non-control rate freezer, the containers being cryopreserved and containing the cells are placed in freezer safe containers which are preferably stored in styrofoam boxes and placed in the freezer for from about 2 to about 24 hours. The containers are thereafter removed from the freezer and immediately quenched in liquid nitrogen for long-term storage. When ready to be used, the cells are thawed in a 37-42° C. water bath and residual cryoprotectant is removed by sequential washings.

When using a control rate freezers freezing profiles are programmed into said freezers to ensure uniform freezing. All such freezing profiles should begin once the sample's temperature reaches minus 4° C. The cooling rates for such control rate temperatures are known to those of skill in the art and U.S. Pat. No. 6,136,525 provides exemplary such cooling rates. Preferably the control rate freezer profile includes a blast of nitrogen that is programmed to compensate for the release of latent heat. Compensating for the release of latent heat decreases the likelihood of cellular damage during cryopreservation of the cell. This programmed cold blast also assists in synchronizing the seeding of external ice on all the freezing containers in a freezing cycle. This is particularly advantageous since the critical point of cryopreserving cells occurs during the ice formation stage. Ice formation begins at a nucleation site which can be a randomly occurring cluster of molecules in the liquid phase. The nucleated ice crystals form into an ice front which expands throughout the liquid until solidification is completed.

A preferred method for cryopreserving cells is to suspend the cells to a density of 2×10⁶ cells/ml in a medium containing 10% DMSO and 10% FCS then dispensing 1 ml of said suspension per vial into 2 ml cryovials. The vials are then frozen at a controlled rate by inserting them into a Nalgene cryo 1° C. freezing container (Cat No. 5100-0001) which is then placed in a −70° C. freezer overnight. The vials are then transferred to the vapor phase of a liquid nitrogen storage tank for long term storage.

Once cells are frozen via the aforementioned freezer techniques, they may be placed in cryogenic storage boxes for long term storage in nitrogen storage freezers. By long term storage, it is contemplated that the cells may be stored for weeks, months or even years prior to resuspension. The freezing containers may be stored in either the vapor or liquid nitrogen phase.

Prior to use, the cells must be thawed and DMSO must be removed. Thawing is accomplished via a 37-42° C. bath. The cells are removed from the container when a slush appears. The cells are then poured into a centrifuge tube containing cold culture media. The initial cell volume is diluted in the centrifuge tube by adding 5 to 10 times the initial cell volume in fresh cold culture media. The suspension is then spun down at 7-15 g for 2 to 5 minutes. The supernatant is aspirated to remove the media containing the residual DMSO. Thereafter, from about 2 to about 4 times fresh media is added to the cell pellet.

The cells of the present invention either before, or after, or before and after cryopreservation are grown and expanded in cell culture. It should be noted, however, that the methods for preparing the cell population of the present invention may omit the cryopreservation and thawing steps.

In preferred embodiments, the cells are grown in a hormonally defined medium as described herein. In one embodiment of this invention, the compositions of this invention are co-culture and used in conjunction with or further comprise feeder cells. Feeder cells provide intracellular matrix and diffusable factors such as growth factors for the growth and expansion of the liver cells. In one embodiment, the feeder cell has little or no ability to be infected with HCV. In another embodiment, the feeder cells are fibroblast cells. In another embodiment, the feeder cells are embryonic mesenchymal fibroblast cells.

Examples of feeder cells according to this invention are mouse embryo fibroblasts (MEF) such as STO cells and rat embryo fibroblasts, e.g., Hogan et al., Manipulating The Mouse Embryo: A Laboratory Manual, 2nd ed. Plainview, N.Y.: Cold Spring Harbor Laboratory Press, 1994; Robertson, E. J. (1987) Teratocarcinomas and Embryonic Stem Cells: A Practical Approach, ed. Robertson, E. J. (IRS, Oxford), pp. 71-112. STO(Reid-99) cells are one type of feeder cells that are useful. STO(Reid-99) cells were deposited on Mar. 27, 2001, in the American Type Culture Collection, 10801 University Boulevard, Manassas, Va. 20110-2209 under the conditions of the Budapest Treaty (ATCC Deposit No: PTA-3236). Methods for culturing and maintaining feeder cells are known in the art. See, for example, in Methods for Tissue Engineering, Ed. Robert Lanza, Academic Press, NY (2002), pp. 151-202.

The feeder cells can be growth-arrested according to methods known in the art. For example, STO cells can be allowed to adhere for 2-48 hours on a cell culture plate. Next, the medium in which the STO cells are incubating would be removed and replaced with medium containing 2 ug/ml Mitomycin C. Then, the STO cells would be incubated at about 37° C. for about 2 hours. After the incubation, the medium containing the Mitomycin C would be removed. The cells would be washed twice, and then the STO cell cultures would be maintained from 0-48 hours before addition of the cell mixtures of this invention.

In one embodiment, the medium used in the resuspension of the cells of the invention is medium that includes free fatty acids (FFA), high density lipoprotein (HDL), and trace elements.

Primary cells, cell lines and tissues of animals or humans can be cultured with a media of this invention. In one embodiment, the culture media comprises serum-free media, calcium, FFA, HDL, nicotinamide, trace elements, EGF, insulin, transferrin and hydrocortisone. According to another embodiment, the culture media can further comprises any one, combination or all of the following ingredients: glucagon, liver growth factor, ethanolamine and thyrotropin releasing factor. In a further embodiment, the culture media does not comprise low density lipoprotein (LDL).

After preparing a cell mixture according to the above process, the cells should be cultured in a media suitable for sustaining the cells and, if necessary, the feeder cells. According to one embodiment of this invention, the media is optimized for a cell mixture that is to be used in an HCV infection. One media useful for this purpose comprises serum free media (e.g., (Dulbecco's modified Eagle's medium (DMEM)) comprising calcium, bovine serum albumin (BSA), free fatty acids (FAA), high density lipoprotein (HDL), nicotinamide, trace elements, epidermal growth factor (EGF), insulin, transferrin, hydrocortisone and optionally, and any one, combination or all of the following ingredients: glucagon, liver growth factor, ethanolamine, and thyrotropin releasing factor. According to one embodiment of this invention, the culturing media does not comprise low density lipoprotein (LDL).

Another media useful for this purpose comprises serum free media comprising, bovine serum albumin (BSA), nicotinamide, epidermal growth factor (EGF), insulin, transferrin, and hydrocortisone.

In one embodiment, the media does not comprise low density lipoprotein (LDL). In another embodiment, the media does not comprise free fatty acids (FAA), high density lipoprotein (HDL), or trace elements. In another embodiment, the media does not contain any one of the ingredients selected from the group consisting of glucagon, liver growth factor, ethanolamine and thyrotropin releasing factor. In another embodiment, the media does not comprise low density lipoprotein (LDL), free fatty acids (FAA), high density lipoprotein (HDL), trace elements, glucagon, liver growth factor, ethanolamine and thyrotropin releasing factor. In another embodiment, the media is serum free.

In one embodiment, the concentration of calcium in the culturing media is between 0.1 mM to 0.6 mM. In another embodiment, the calcium concentration is approximately 0.5 mM. In one embodiment, the concentration of the BSA is 500 ug/ml. In another embodiment, the concentration of the nicotinamide is 5 mM. In one embodiment, the concentration of the insulin is 10 ng/ml. In one embodiment, the concentration of the free fatty acids is 7.6 uEq/L. In one embodiment, the concentration of EGF is 100 ng/ml. In one embodiment, the concentration of the liver growth factor is 20 ug/ml. In one embodiment, the concentration of the ethanolamine is 10⁻⁶M. In one embodiment, the concentration of the thyrotropin releasing factor is 10⁻⁶M. In one embodiment, the concentration of the HDL is 5 ug/ml. In one embodiment, the concentration of the hydrocortisone is 10⁻¹⁰M.

In one embodiment, the media is IM-HDM media, which comprises DMEM (high glucose), 500 ug/ml BSA, 7.6 uEq/L free fatty acids (FAA), 5 ug/ml HDL, 5 mM nicotinamide, 1× trace elements [1×10⁻⁷M copper, 5×10⁻¹¹M zinc, 3×10⁻¹⁰M selenium], 100 ng/ml EGF, 10 ng/ml insulin, 5 ug/ml transferrin, 10⁻⁶M hydrocortisone, 2 ug/ml glucagon, 20 ug/ml liver growth factor, 10⁻⁶M ethanolamine, 10⁻⁶M thyrotropin releasing factor]. In one embodiment, the 7.6 uEq/L of total FFAs comprises a mixture 2.36 μM palmitic acid(16:0), 0.21 uM palmitoleic acid(cis-16:1 n-7), 0.88 μM steric acid(18:0), 1.02 μM oleic acid(cis-18:1 n-9), 2.71 μM linoleic acid(cis-18:2 n-6), and 0.43 μM linolenic acid(cis 18:3 n-3). The media can also comprise antibiotics to deter bacterial growth, for example, 1× penicillin/streptomycin.

In another embodiment, the media comprises serum free media comprising, bovine serum albumin (BSA), nicotinamide, epidermal growth factor (EGF), insulin, transferrin, and hydrocortisone.

The cell mixtures of the compositions of this invention can be plated on plastic substrates coated with extracellular matrix. Examples of extracellular matrix components include, but are not limited to collagen, such as, for example, collagen Type I, collagen Type IV, or the adhesion proteins, fibronectin and laminin, or Matrigel (ICN Biochemicals Inc.). The collagen, when employed, can be used alone or in combination with laminin or fibronectin, or in combination with proteoglycans, or with tissue extracts enriched in extracellular matrix materials. Extracellular matrixes can also be provided by the feeder cells describe above. Such cellular mixtures and extracellular matrix combinations can be used in HCV assay methods according to this invention.

The cell compositions of the present invention will be useful in providing in vitro cultures that can be infected with HCV and therefore provide useful in vitro culture systems for the identification and rapid screening of compounds that may be useful for treating patients suffering from HCV and/or for research relating to HCV infection. Thus, in preferred embodiments, the cell compositions of this invention can be contacted with RNA898 or an HCV infectious equivalent of RNA898. An RNA898 infectious equivalent is an HCV strain, other than RNA898 that is capable of producing greater than about 5,000 copies, greater than about 10,000 copies or greater than about 50,000 copies of HCV RNA at seventy-two hours after contacting 4×10⁴ cells of the compositions prepared according to the methods of this invention with said HCV virus. According to one embodiment, the cells are infected by contacting the composition of this invention with RNA898 or its infectious equivalent for about 24 hours at about 37 degrees C. in a volume of about 0.52 ml per cm². According to one embodiment of this invention, the cells being infected with HCV are cultured with an extracellular matrix. In another embodiment of this invention, the extracellular matrix is provided by feeder cells (e.g., STO-(Reid-99) cells).

The amount of HCV produced from the cells in the compositions of this invention can be determined by measuring, e.g., HCV protein or nucleic acid production. For example, the number of copies of HCV RNA found associated with the cells (i.e., in or attached thereto) and/or in the media in which the cells are cultured can be quantified. There are techniques known in the art that can be used for observing whether HCV protein or nucleic acid molecules have been produced. For example, western blot of the proteins probed with antibodies directed against HCV proteins or blots of gels probed labeled nucleic acids molecules that are complementary to HCV nucleic acid sequence. Methods for extracting protein and nucleic acid molecules from cells and cell culture media are well known in the art and such kits for this purpose are commercially available.

For quantifying with greater accuracy the number of copies of HCV particles produced according to this invention, reverse-transcriptase polymerase chain reaction (RT-PCR) is useful. According to one embodiment of this invention, the RT-PCR method is modified such that the number of copies of HCV RNA are determined by comparing its value to a second nucleic acid molecule of known amount that is added to the samples of cells, cell extracts and/or media to be assayed either in the form of a second virus or a second nucleic acid molecule. It is desirable that the second virus is closely related to HCV or that the second nucleic acid molecule is closely related to HCV RNA (i.e., similar in length, in nucleic acid composition and in viral capsid structure). In one embodiment, the second nucleic acid molecule is in a flavivirus capsid. In one embodiment, the second RNA molecule is the RNA from Bovine Viral Diarrhea Virus (“BVDV”), e.g., the BVDV NADL strain (ATCC Deposit No: VR-534).

The presence of the second virus or nucleic acid molecule is advantageous in that it serves as an internal control for the quantification of the first nucleic acid molecule. This internal control allows for the monitoring and correction of random fluctuations and assay variability.

For example, the present invention provides the method comprising the steps of:
1. combining said HCV with a known amount of Bovine Viral Diarrhea Virus (“BVDV”), wherein said BVDV contains a second nucleic acid molecule with a composition of this invention;
2. extracting from the cells of the composition or the media in which the cells are cultivated a first nucleic acid molecule derived from HCV and said second nucleic acid molecule derived from BVDV to form a combined nucleic acid extract;
3. adding to said combined nucleic acid extract a first detectable probe, which is specific for said first nucleic acid and a second detectable probe, which is specific for said second nucleic acid;
4. amplifying said combined nucleic acid extract by PCR means;
5. quantifying at various cycles during said amplification a detectable signal released independently from said first detectable probe and said second detectable probe;
6. extrapolating the results of step (e) to calculate the amount of said first nucleic acid molecule in said HCV and the amount of said second nucleic acid molecule in BVDV; and
7. evaluating the accuracy of said calculated amount of said first nucleic acid molecule determined in step (f) by comparing said calculated amount of said second nucleic acid in step (f) with said known amount of said second nucleic acid used in step (a).

According to another embodiment, the above method comprises the additional step of adjusting said calculated amount of said first nucleic acid determined in step (f) by a factor determined by comparing said calculated amount of said second nucleic acid in step (f) with said known amount of said second nucleic acid used in step (a).

According to another embodiment, the present invention provides a method of determining the effect of a compound on the production of an HCV, comprising the steps of adding a compound before or after administering the HCV to the compositions of this invention and subsequently determining the presence of HCV associated with the cells in the compositions and/or media in which the infected cells are cultivated. If it is desired that the compound is administered after the HCV is contacted with the composition, then it is preferable that the compound be administered within 10 days after the HCV is contacted with the composition. The compounds to be tested according to this invention can inhibit or activate the production of HCV. Accordingly, a compound can inhibit any stage of the life cycle of the HCV to achieve its effect. Examples of such compounds include, but are not limited to, synthetic or purified chemical compounds, proteins and nucleic acid molecules. The samples to which the compounds were added can be compared to other samples treated under the same conditions but have not been exposed to the compound or have been exposed to another compound that is known to have little or no effect on HCV production.

It is contemplated that the cell populations of the present invention will be employed in in vitro screening assays in which agents that affect HCV infection of liver cells will be monitored. Such methods may be performed in multiwell (e.g., 96-well) plates. The assays will involve incubating the cell populations of the present invention, optionally, together with a feeder layer in a suitable growth medium and suitable container. The cells are then contacted with the appropriate HCV as discussed above. The infection of the cells by the HCV in the presence and absence of test compound will be assessed. It is contemplated that various concentrations of the test compound being tested may be added to co-culture of cells, in the presence and absence of the virus. Furthermore, the cells may be exposed to the test compound at any given phase in the growth cycle of the virus. For example, in some embodiments, it may be desirable to contact the virus particles with the compound at the same time as a viral infection of the cells is initiated (i.e., at the same time that the virus is added). Alternatively, it may be preferable to add the compound at a later stage in the viral life-cycle (i.e., after the cells have become infected with the virus. In still other embodiments, the cells may be contacted with the test compound prior to addition of the virus in order to determine whether the test compound has a prophylactic effect against infection by the virus. Determining the particular stages of the virus life cycle are in is achieved through methods well known to those of skill in the art.

The varying concentrations of the given test compound are selected with the goal of including some concentrations at which no toxic effect is observed and also at least two or more higher concentrations at which a toxic effect is observed. For example, assaying several concentrations of the test compound within the range from 0 micromolar to about 300 micromolar is commonly useful to achieve these goals. It will be possible or even desirable to conduct certain of these assays at concentrations higher than 300 micromolar, such as, for example, 350 micromolar, 400 micromolar, 450 micromolar, 500 micromolar, 600 micromolar, 700 micromolar, 800 micromolar, 900 micromolar, or even at millimolar concentrations. The estimated therapeutically effective concentration of a compound provides initial guidance as to upper ranges of concentrations to test.

In an exemplary set of assays, the test compound concentration range under which the assay is conducted comprises dosing solutions which yield final test compound assay concentrations of 0.05 micromolar, 0.1 micromolar, 1.0 micromolar, 5.0 micromolar, 10.0 micromolar, 20.0 micromolar, 50.0 micromolar, 100 micromolar, and 300 micromolar of the compound in assay medium. As mentioned, these are exemplary ranges and it is envisioned that any given assay will be run in at least four different concentrations, more preferably the concentration dosing will comprise, for example, 4, 5, 6, 7, 8, 9, 10, 11, 12, 13, 14, 15 or more concentrations of the compound being tested. Such concentrations may yield, for example, a media concentration of 0.05 micromolar, 0.1 micromolar, 0.5 micromolar, 1.0 micromolar, 2.0 micromolar, 3.0 micromolar, 4.0 micromolar, 5.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 55.0 micromolar, 60.0 micromolar, 65.0 micromolar, 70.0 micromolar, 75.0 micromolar, 80.0 micromolar, 85.0 micromolar, 90.0 micromolar, 95.0 micromolar, 100 micromolar, 110.0 micromolar, 120.0 micromolar, 130.0 micromolar, 140.0 micromolar, 150.0 micromolar, 160.0 micromolar, 170.0 micromolar, 180.0 micromolar, 190.0 micromolar, 200.0 micromolar, 210.0 micromolar, 220.0 micromolar, 230.0 micromolar, 240.0 micromolar, 250.0 micromolar, 260.0 micromolar, 270.0 micromolar, 280.0 micromolar, 290.0 micromolar, and 300 micromolar in the test composition.

The compounds to be tested may include fragments or parts of naturally-occurring compounds or may be derived from previously known compounds through a rational drug design scheme. It is proposed that compounds isolated from natural sources, such as animals, bacteria, fungi, plant sources, including leaves and bark, and marine samples may be assayed as candidates for the presence of potentially useful pharmaceutical compounds. Alternatively, pharmaceutical compounds to be screened for toxicity could also be synthesized (i.e., man-made compounds).

The types of compounds being monitored may be antiviral compounds, antibiotics, anti-inflammatory compounds, antidepressants, analgesics, antihistamines, diuretic, antihypertensive compounds, antiarrythmia drugs, chemotherapeutic compounds for the treatment of cancer, antimicrobial compounds, among others.

Regardless of the source or type of the compound to be tested for cytotoxicity, it may be necessary to monitor the biological activity of the compounds to provide an indication of the therapeutic efficacy of a particular compound or group of compounds. Of course, such assays will depend on the particular therapeutic indication being tested. Exemplary indications include HCV or other viral infection.

In preferred embodiments, the assays of the present invention may be used as part of a drug discovery program to identify a putative therapeutic compound with increased efficacy against HCV infection. Drug discovery begins with the identification of a range of candidate substances that show promise in a targeted therapeutic area. In exemplary embodiments, an exemplary core or template structure such as VX-950 may be used for future drug development efforts. Other structures may likewise provide useful, particularly structures designed as anti-HCV agents through rational drug design. Once the template is selected, additional chemistry and structure activity analyses are performed to increase the potency of the compound. This process yields the lead compounds. A screen using the methods of the invention at this stage of the process may be performed to provide efficacy data on these potential lead compounds. The top lead compounds are selected to enter preclinical animal testing. Incorporation of the present selection methods early in the discovery process should greatly reduce the number of compounds that fail during this late stage.

The in vitro screening using the cell populations of the present invention may be employed at any stage in the drug discovery program but may prove especially valuable early in the discovery process. The information obtained from such analysis provides the chemists with the appropriate information to maximizing potency and efficacy in the new templates. Using these methods, the putative therapeutic compounds can be ranked or prioritized based on their relative binding efficacies and compared to known drugs in the same therapeutic and chemical class. For example, the VX-950 could be used as a reference compound for new anti-HCV agents.

### Structure of VX-950

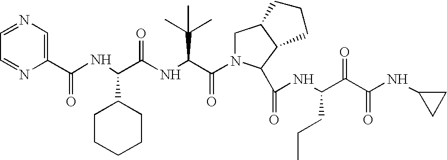

High throughput assays for screening numerous compounds for efficacy using the cell populations of the present invention are specifically contemplated. In certain embodiments, the high throughput screens may be automated. In high throughput screening assays, groups of compounds are exposed to cell cultures comprised of the cells of the present invention that have been infected with HCV and similar cultures that have not been exposed to HCV. These groups of compounds may be assembled from collections of compounds previously individually prepared and since stored in a compound bank, the assembly being random or guided by the use of similarity programs from which similar structures are formed.

In addition, there has also been a rapid growth in the deliberate preparation and use of libraries and/or arrays of compounds. Each library contains a large number of compounds which are screened against a biological target such as an enzyme or a receptor. When a biological hit is found, the compound responsible for the hit is identified. Such a compound, or lead, generally exhibits relatively weak activity in the screen but forms the basis for the conduct of a more traditional medicinal chemistry program to enhance activity. The libraries may be prepared using the rapidly developing techniques of combinatorial chemistry or by parallel synthesis (DeWitt et al., Proc Natl Acad Sci, 90, 6909, 1993; Jung et al., Angew Chem Int Ed Engl, 31:367-83, 1992; Pavia et al., Bioorg Med Chem Lett, 3:387-96, 1993).

Alternatively, the compounds to be screened may be from a library based upon a common template or core structure such as e.g., the VX-950 structure described above. Such techniques are described in e.g., WO 95/32184 (oxazolone and aminidine template), WO 95/30642 (dihydrobenzopyran template) and WO 95/35278 (pyrrolidine template). The template will have a number of functional sites, for instance three, each of which can be reacted, in a step-wise fashion, with a number of different reagents, for instance five, to introduce 5×5×5 different combinations of substituents, giving a library containing 125 components. The library will normally contain all or substantially all possible permutations of the substituents. The template may be a ‘biased’ template, for instance incorporating a known pharmacophore such as a benzodiazepine ring or an ‘unbiased’ template, the choice of which is influenced more by chemical than biological considerations.

Thus, the cell populations and the methods of the present invention may be used to identify lead compounds for drug discovery. In addition to the library screening discussed above, such lead compounds may be generated by random cross screening of single synthetic compounds made individually in the laboratory or by screening extracts obtained from natural product sources such as microbial metabolites, marine sponges and plants.

In another alternative, the compounds may be generated through rational drug design based on the structure of known biologically active compounds and/or their sites of biological action. This has now been complemented by the powerful techniques of computer-assisted drug design. The goal of rational drug design is to produce structural analogs of biologically active molecules of interest. Such technologies will yield potentially thousands of compounds for a particular indication that may be screened for anti-HCV activity using the present invention.

According to one embodiment, the above method is used to simultaneously screen the affect of a plurality of compounds on HCV production. For example, each well of a 96-well plate could contain a different compound to be screened according to the methods of this invention. In a further embodiment, the methods of this invention are used to identify compounds that inhibit the production of HCV.

In one embodiment, the primers and probe used in the methods of this invention are designed based upon most conserved regions of HCV strains. The probe can also be constructed based upon the following additional criteria: a) the melting temperature of the probe is 8° C. to 10° C. higher than that of the primers; b) no G's are present at the 5′ end; c) there is not a stretch of more than 4 G's; and/or d) the probe does not form internal structures with high melting temperatures or form a duplex with itself or with any of the primers. In one embodiment, the entire PCR region was about 150 base pairs in length.

Useful primers and probe for the 5′ UTR of BVDV can be designed based on the same set of criteria. In addition, care was taken to ensure that the primers or probe of HCV has the least amount of homology to those of BVDV. Primers and probes can be obtained from commercial sources that synthesize and prepare modified nucleic acid molecules (e.g., Oligo and PE Applied Biosystems). BVDV can be maintained by infection of MDBK cells.

In one embodiment of the invention, two different dual labeled fluorogenic probes are used, each specific for one but not the other of the HCV nucleic acid molecules and the second nucleic acid molecules. In a further embodiment, each fluorogenic probe typically has a reporter dye at the 5′ end and a quencher dye at the 3′ end. The two different fluorogenic probes are selected such that they give distinct fluorescence peaks that can be detected without cross interference between the two peaks. For example, as discussed supra, the 5′ end of the first detectable probe can be labeled with a reporter dye such as 6 carboxy fluorescein (“6 FAM”), and the 5′ end of the second detectable probe can be labeled with a reporter dye such as VIC. The 3′ end of both detectable probes can be labeled with a quencher dye such as 6 carboxymethyl rhodamine (“6 TAMRA”). Thus, when bound to the first nucleic acid and the second nucleic acid, the proximity of the reporter dye at the 5′ end to the quencher dye at the 3′ end of the probe results in a suppression of the fluorescence. During amplification, when the Tth polymerase moves along the nucleic acid sequence, the quencher is removed from the probe by the action of the 5′ 3′ exo, thereby degrading the fluorogenic probe. This results in a fluorescence emission, which is recorded as a function of the amplification cycle. Thus, monitoring the fluorescence emission provides a basis for measuring real time amplification kinetics.

Examples of useful primers and probes for HCV genotype 1 are: (SEQ ID NO:1) 5′CCATGAATCACTCCCCTGTG 3′ (forward primer), (SEQ ID NO:2) 5′ CCGGTCGTCCTGGCAATTC 3′ (reverse primer), and the HCV probe, (SEQ ID NO:5) 5′6 FAM-CCTGGAGGCTGCACGACACTCA-TAMRA 3′. The primers and probe for BVDV comprised the forward primer, (SEQ ID NO:3) 5′-CAGGGTAGTCGTCAGTGGTTCG-3′, the reverse primer, (SEQ ID NO:4) 5′-GGCCTCTGCAGCACCCTATC-3′, and the probe, 5′ VIC (SEQ ID NO: 6)-CCCTCGTCCACGTGGCATCTCGA-TAMRA 3′.

The RT and the PCR reactions can be carried in the same wells of a 96 well plate optical tray with caps (PE Applied Biosystems, Foster City, Calif.). In one embodiment of this invention, a multiplex RT-PCR reaction is used (i.e., a RT-PCR reaction that amplifies and measures two different RNA species simultaneously, e.g., HCV RNA and BVDV RNA, in the same tube). The multiplex reactions has the advantage of allowing the practitioner to determine if an HCV negative result was due to the fact that the culture was truly negative or some technical failure in the extraction or RT-PCR steps. Ten or twenty ul of viral RNA or RNA standard can be amplified in a 50 ul RT-PCR reaction with 1× Taqman EZ buffer (PE Applied Biosystems), 3 mM manganese acetate, 300 mM each of dATP, dCTP, dGTP, and dUTP, 5 units Tth polymerase (Epicentre), 4.0% enhancer (Epicenter), some concentration of probes and primers. The Taqman RT-PCR assay can be run for 25 min at 60° C. (RT), 5 min at 95° C., and followed by 45 cycles of two-step PCR reaction (60° C. for 1 min and 95° C. for 15 sec). For an assay with HCV and another nucleic acid (the multiplex Taqman assay), the amount of HCV and BVDV primers can be optimized using a matrix mixture of various concentration of both sets of primers. The final assay condition includes 200 nM of both 6-FAM-labeled HCV probe and VIC-labeled BVDV probe, 400 nM of both HCV primers, and 45 nM of both BVDV primers.

Throughout the specification and claims, the word “comprise,” or variations such as “comprises” or “comprising,” will be understood to imply the inclusion of a stated integer or group of integers but not the exclusion of any other integer or group of integers.

U.S. provisional application 60/279,174, filed Mar. 27, 2001, and U.S. Patent Application No. 20020142449 and articles recited herein are incorporated by reference.

While a number of embodiments of this invention have been presented, it is apparent that the basic construction can be altered to provide other embodiments which utilize the compositions and methods of this invention. Therefore, it will be appreciated that the scope of this invention are to be defined by the claims and specification rather than the specific embodiments which are exemplified here.

General methods related to the practice of this invention may be found in WO 02/077206. For general methods for hepatocyte isolation and cryopreservation, see: Mitry, R. R., Hughes, R. D., and Dhawan, A. (2002). Progress in human hepatocytes: isolation, culture & cryopreservation. Cell & Developmental Biology 13, 463-467. For a detailed method for fetal hepatocyte isolation (from rats), see: Arahyetes, R. M., Sierra, E., Codesal, J., Barrutia, M. S. G., Arza, E., Cubero, J., Ortiz, A., and Magnanto, P. (2001). Optimization of the technique to isolate fetal hepatocytes, and assessment of their functionality by transplantation. Life Sciences 68, 763-772.

Once the screening assays identify an appropriate anti-HCV agent the agent may be formulated as a pharmaceutical composition and may further be tested in in vivo models for HCV infection.

In certain aspects of the present invention, all the necessary components for conducting the selection and screening assays may be packaged into a kit. Specifically, the present invention provides a kit for use in such an assay comprising a packaged set of reagents for conducting the assay as well as test or reference compounds, instructions packaged with the reagents for performing one or more variations of the assay of the invention using the reagents. The instructions may be fixed in any tangible medium, such as printed paper, or a computer-readable magnetic or optical medium, or instructions to reference a remote computer data source such as a world wide web page accessible via the internet.

All of the documents cited herein are incorporated herein by reference.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cryopreservation

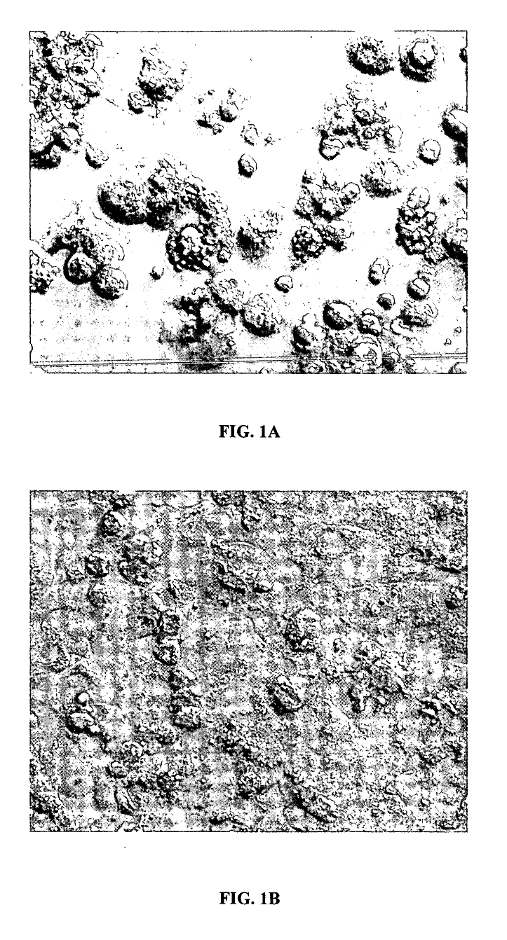

Human fetal liver cells were isolated by collagenase digestion, filtration through a 70 um filter and three low speed centrifugations. The cells were suspended to 2×10⁶/ml in DMEM containing 10% FCS and 10% DMSO the frozen by controlled rate freezing. A vial of cells was thawed by immersion in a 37° C. waterbath, suspension in DMEM containing 10% FCS, pelleted by low speed centrifugation, resuspended in 10 ml of DMEM 10% FCS and allowed to attach overnight to collagen coated plastic. Non-adherent cells were removed by gentle rinsing of the monolayer then the medium was replaced by hormonally defined medium. The micrograph was taken with a 10× objective and Hoffman Modulation Contrast optics. See FIG. 1.

### Example 2

### Time Course Analysis

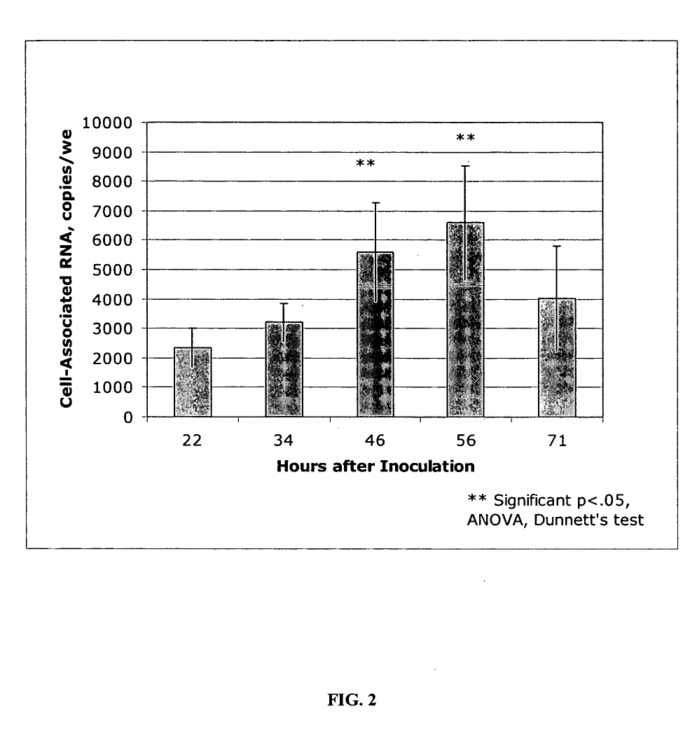

Cryopreserved human fetal liver cells were thawed and allowed to adhere overnight to wells of a collagen coated 96-well plate. Non-adherent cells were removed and medium replaced with hormonally defined medium containing DMEM, BSA, nicotinamide, EGF, insulin, transferring, and hydrocortisone (HDM2). The wells were inoculated with HCV virus and incubated for 8 hours at 37° C. The non-adsorbed virus was removed, the cultures washed twice with HDM2, then fresh HDM2 was added. After incubation for the indicated (from start of inoculation) 4 wells were rinsed twice with PBS then the cells lysed with chaotropic buffer and RNA isolated using the Qiagen 96-well RNeasy procedure. HCV RNA was quantitated using the multiplex RT-PCR method and the mean and standard deviation of the quadruplicate samples (HCV copy number per well) are presented. Analysis of the results with Dunnett's test indicated samples harvested 46 and 56 hours after inoculation contained significantly more HCV RNA than the earlier time point at 22 hours. See FIG. 2.

### Example 3

### Inhibition of HCV Infection

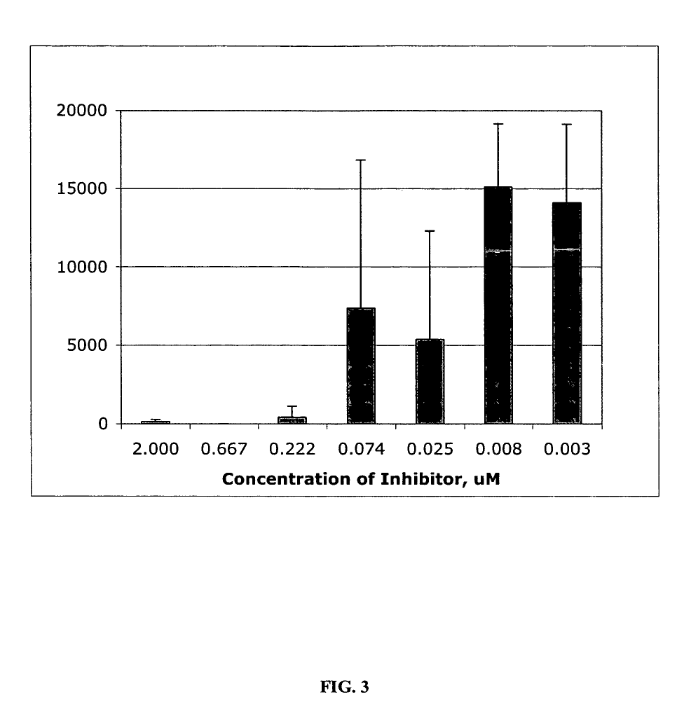

Cryopreserved human fetal liver cells were thawed and allowed to adhere to wells of a collagen coated 96-well plate for 6 hours at 37° C. Non-adherent cells were removed and medium replaced with hormonally defined medium containing DMEM, BSA, nicotinamide, EGF, insulin, transferrin, and hydrocortisone (HDM2). The wells were inoculated with HCV in the presence of different concentrations of VX-950 and incubated for 15 hours at 37° C. The non-adsorbed virus was removed, the cultures washed twice with DMEM, then fresh HDM2 containing the same concentration of VX-950 was added. After incubation for an additional 47 hours, the wells were rinsed twice with PBS then the cells lysed with chaotropic buffer and RNA isolated using the Qiagen 96-well RNeasy procedure. HCV RNA was quantitated using the multiplex RT-PCR method and the mean and standard deviation of the triplicate samples at each concentration of inhibitor are presented (HCV copy number per well). Concentrations of VX-950 of 0.2 uM or greater showed nearly complete inhibition of HCV replication. See, FIG. 3.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

1. A composition comprising a cryopreserved cell mixture that comprises liver cells and hematopoietic cells isolated from the liver of a human aged three months or older after conception, wherein a preparation that comprises 4×10⁴ thawed cells of said cryopreserved cell mixture in the presence of a feeder cell line in a growth media produces more than about 5000 copies of hepatitis C virus (HCV) RNA in the media seventy two hours after administration of HCV virus RNA898 to said preparation.

2. (canceled)

3. (canceled)

4. (canceled)

5. (canceled)

6. A composition comprising a cell mixture prepared by the following steps:
(a) obtaining a suspension of liver cells from a human aged three months or older after conception in a buffer comprising EGTA by treating said liver cells with a protease, wherein said suspension of liver cells has been treated to remove objects that are 40 micron or greater and said suspension of liver cells does not contain red blood cells;
(b) resuspending the cells of step (a) in a media comprising BSA, nicotinamide, epidermal growth factor (EGF), insulin, transferrin and hydrocortisone and optionally, any one of the ingredients selected from the group consisting of glucagon, liver growth factor, ethanolamine and thyrotropin releasing factor; and
(c) culturing the cells in the serum free media of step (b).

7. The method of claim 6 further comprising:
(d) cryopreserving the cells of step (a) in suspension that comprises 10% DMSO and 10% fetal calf serum prior to step (b); and
(e) thawing the composition of step (d) prior to the resuspension in step (b).

8. (canceled)

9. (canceled)

10. (canceled)

11. (canceled)

12. A composition comprising a cell mixture prepared by the following steps:
(a) dissecting a liver of a human aged three months or older after conception in a buffer comprising EGTA;
(b) incubating the dissected liver in a buffer comprising collagenase to separate cells from the liver;
(c) remove objects 40 micron or greater from the separated cells;
removing red blood cells from the separated cells;
(e) resuspending the cells in 10% DMSO and 10% fetal calf serum;
(f) cryopreserving the composition of step 5;
(g) thawing the composition of step 6;
(h) resuspending the cells of step (f) in a serum free media, comprising calcium, free fatty acids (FFAs), high density lipoprotein (HDL), nicotinamide, trace elements, epidermal growth factor (EGF), insulin, transferrin and hydrocortisone and optionally, any one of the ingredients selected from the group consisting of glucagon, liver growth factor, ethanolamine and thyrotropin releasing factor; and
(i) culturing the cells in the serum free media of step (e).

13. A composition comprising a cell mixture prepared by the following steps:
(a) dissecting a liver of a human aged three months or older after conception in a buffer comprising EGTA;
(b) incubating the dissected liver in a buffer comprising collagenase to separate cells from the liver;
(c) remove objects 40 micron or greater from the separated cells;
(d) removing red blood cells from the separated cells;
(e) resuspending the cells of step (d) in a media comprising BSA, nicotinamide, epidermal growth factor (EGF), insulin, transferrin and hydrocortisone and optionally, any one of the ingredients selected from the group consisting of glucagon, liver growth factor, ethanolamine and thyrotropin releasing factor; and
(f) culturing the cells in the serum free media of step (e).

14. A composition according to claim 13, comprising the further steps between steps (d) and (e):
(g) resuspending the cells of step (d) in 10% DMSO and 10% fetal calf serum;
(h) cryopreserving the composition of step g; and
(i) thawing the composition of step h.

15. (canceled)

16. (canceled)

17. (canceled)

18. A composition comprising an expanded mixed cell population of liver cells and hematopoietic cells released from the liver of a human aged three months or older after conception, wherein said mixed cell population is selected to exclude cells that express CD34+ but includes cells that express alpha fetoprotein, albumin, and glycophorin.

19. (canceled)

20. (canceled)

21. (canceled)

22. (canceled)

23. (canceled)

24. (canceled)

25. (canceled)

26. (canceled)

27. The composition according claim 1, further comprising an HCV.

28. The composition according to claim 27, wherein the HCV is RNA898.

29. A composition comprising a media, BSA, nicotinamide, EGF, insulin, transferrin, and hydrocortisone, and, optionally, not low density lipoprotein (LDL) and, optionally, not any one of the ingredients selected from the group consisting of glucagon, liver growth factor, ethanolamine and thyrotropin releasing factor.

30. (canceled)

31. A method for isolating and cultivating a cell mixture comprising the steps of:
(a) obtaining a suspension of liver cells from a human aged three months or older after conception in a buffer comprising EGTA by treating said liver cells with a protease, wherein said suspension of liver cells has been treated to remove objects that are 40 micron or greater and said suspension of liver cells does not contain red blood cells;
(b) resuspending the cells of step (a) in a media comprising BSA, nicotinamide, EGF, insulin, transferrin, hydrocortisone; and
(c) culturing the cells in the serum free media of step (b).

32. The method according to claim 31, comprising the further steps between steps a) and b):
(d) cryopreserving the cells of the suspension of step (a) in a composition comprising 10% DMSO and 10% fetal calf serum; and
(e) thawing the cryopreserved cells of step (d) prior to step (b).

33. The method of claim 31, wherein said suspension of liver cells are obtained in step (a) by dissecting a liver of a human aged three months or older after conception in a buffer comprising EGTA.

34. The method of claim 33, wherein said buffer comprising EGTA is supplemented or replaced with a protease containing buffer wherein said protease is selected from the group consisting of collagenase, trypsin, pronase, and dipase.

35. The method of claim 33, said buffer comprising EGTA is supplemented or replaced with a protease containing buffer wherein said buffer comprises a cocktail of two or more proteases selected from the group consisting of collagenase, trypsin, pronase, and dipase.

36. The method of claim 34 wherein said buffer comprising EGTA further comprises an enzyme to breakdown the carbohydrate components of the tissue, said enzyme selected from the group consisting of hyaluronidase, and neuraminadase.

37. The method according to claim 31, wherein the red blood cells are removed by centrifuging the suspended cells at low speed (50×g) to pellet the larger cells for approximately 3 to 4 minutes, washing the cells that pellet and repeating the centrifuging and washing steps.

38. The method according to claim 31, wherein the media does not contain low density lipoprotein (LDL); and/or media does not contain calcium, FFA, HDL, or trace elements; and/or media does not contain glucagon, liver growth factor, ethanolamine or thyrotropin releasing factor; and/or the media is not serum free media.

39. A method for infecting cells with HCV comprising the step of contacting the composition of any one of claims 1 to 29 or the cell mixture prepared according to claim 31 with the HCV virus RNA898 or its infectious equivalent.

40. The method according to claim 39, wherein the HCV virus is added to composition and incubated for about 24 hours at about 37° C. in a volume of about 0.52 ml per cm² prior to washing the cells in the composition.

41. A method for assaying HCV infection comprising the steps of:
(a) incubating the composition according to claim 1, with a feeder cell;
(b) contacting the cells in the composition with RNA898 or its infectious equivalent; and
(c) measuring the presence of the HCV RNA associated with the cells of the composition, the media in which the cells are cultured or both the cells and the media.

42. The method according to claim 41, wherein the feeder cell is the STO(Reid 99) cell.

43. The method according to claim 41, wherein the quantity of HCV RNA is measured by comparing (a) the amount of HCV RNA present associated with the cells or media in which the cells are cultivated with (b) an amount of RNA from a second virus that is used as an internal control.

44. The method according to claim 43, wherein the second viral RNA is from Bovine Viral Diarrhea Virus (BVDV).

45. A method for evaluating the ability of a compound to affect the production of an HCV comprising the steps of:
(a) incubating the composition according to claim 1, with a feeder cell;
(b) contacting the cells in the composition with RNA898;
(c) administering the compound to the composition before or after the cells are contacted with RNA898 or its infectious equivalent; and
(d) measuring the HCV associated with the cells, the media in which the cells are cultured or both the cells and the media.

46. The method according to claim 45, wherein the compound inhibits HCV production.

47. The method according to claim 45, wherein a plurality of compounds are screened simultaneously for their ability to inhibit HCV production.

48. The method according to claim 45, comprising the further step of comparing the measurement of step (d) with the amount of HCV associated with control cells, the media in which the control cells are cultured or both the control cells and the media, wherein the control cells have been subjected to steps (a)-(d) except that no compound or a known inactive compound has been administered.

49. The method according to claim 45, wherein the feeder cell is the STO(Reid 99) cell.

50. The method according to claim 45, wherein the presence of HCV is measured by determining the quantity of HCV RNA associated with the cells or media in which the cells are cultivated.

51. The method according to claim 45, wherein the quantity of HCV RNA is determined by reverse transcriptase polymerase chain reaction (RT PCR).

52. The method according to claim 45, wherein the quantity of HCV RNA is measured by comparing (a) the amount of HCV RNA associated with the cell or the media in which the cells are cultivated with (b) an amount of RNA from a second virus that is used as an internal control.

53. The method according to claim 45, wherein the second virus is Bovine Viral Diarrhea Virus (BVDV).

54. A method of preparing a mixed population of liver cells comprising:
(a) obtaining a suspension of liver cells from a human aged three months or older after conception in a buffer comprising EGTA by treating said liver cells with a protease, wherein said suspension of liver cells has been treated to remove objects that are 40 micron or greater and said suspension of liver cells does not contain red blood cells;
(b) isolating CD34−/alpha fetoprotein+/glycoprotein+/albumin+cells from said suspension of liver cells;
(c) resuspending the cells of step (b) in a media comprising BSA, nicotinamide, epidermal growth factor (EGF), insulin, transferrin and hydrocortisone and optionally, any one of the ingredients selected from the group consisting of glucagon, liver growth factor, ethanolamine and thyrotropin releasing factor; and
(d) co-culturing said cells from step (c) in culture media comprising feeder cells to expand said population of liver cells.

55. The method of claim 54 comprising cryopreserving the cells of the suspension of step (a) or the cells isolated after step (b) in a composition comprising 10% DMSO and 10% fetal calf serum; and thawing said cryopreserved cells prior to step (c).

56. (canceled)
